(19) United States
(12) Patent Application Publication
(10) Pub. No.: US 2025/0186722 A1
(43) Pub. Date: Jun. 12, 2025

(54) ARRANGEMENT FOR NOISE REDUCTION

(71) Applicant: Drägerwerk AG & Co. KGaA, Lübeck (DE)

(72) Inventor: René-Christian CASPARY, Lübeck (DE)

(21) Appl. No.: 18/976,718

(22) Filed: Dec. 11, 2024

(30) Foreign Application Priority Data
Dec. 12, 2023 (DE) 10 2023 134 721.7

(51) Int. Cl. A61M 16/00

(52) U.S. Cl. CPC A61M 16/0066; A61M 16/0003; A61M 2205/42

(57) ABSTRACT

An arrangement (1) reduces noise at a gas inlet (8) of a forced-air (blower) ventilator (6). One or more labyrinth elements (2) in combination with a connecting element (3) makes it possible to effectively reduce the emission of operating noises from the blower ventilator (6) to the environment (5).

### CROSS REFERENCE TO RELATED APPLICATIONS

This application claims the benefit of priority under 35 U.S.C. § 119 of German Application 10 2023 134 721.7, filed Dec. 12, 2023, the entire contents of which are incorporated herein by reference.

### TECHNICAL FIELD

The present invention pertains to an arrangement for reducing noise at a gas inlet of a blower ventilator (forced-air breathing apparatus). Such arrangements may also be referred to as silencers or inlet silencers or mufflers.

### BACKGROUND

Silencers are known from many areas of technology, for example to reduce intake noise on a combustion engine, for example for motor vehicles, ships or also for stationary engines in combined heat and power plants.

Vacuum cleaners sometimes also have components for sound attenuation. Sound attenuation in or at the inlet area of blowers is essential to reduce sound emissions even in the low load range or partial load range.

The state of the art provides possibilities for reducing noise or sound emissions from ventilators.

For example, US2008257347 AA shows sound insulation formed by lining with acoustic foam.

Configurations consisting of acoustic foam in the inlet area can also be realized, as shown, for example, by EP 2739857 B1.

For longer-term operation of blower ventilators in hospitals, for example for more than 5 to 10 years, elements with acoustic foam may require regular maintenance to check these elements for hygiene and/or material ageing and, if necessary, to replace them when the maximum period of use has been reached or the mechanical stability of the foam structure has deteriorated.

From the point of view of people (individuals) who have to ensure the operational readiness of the blower ventilator as maintenance personnel, such a replacement of components is associated with time planning, logistics in the technical area of maintenance and also in the commercial area, for example in purchasing.

### SUMMARY

The invention is based on the motivation to develop a blower ventilation device that is as free as possible from a large number of maintenance or replacement parts and, in particular, can be configured without elements with acoustic foam.

In the light of the state of the art, it is an object of the present invention to provide a means to reduce noise of a blower ventilator (forced-air breathing apparatus).

The object of specifying a noise reduction for a blower ventilator is attained by an arrangement for reducing noise with features according to the invention.

Advantageous embodiments of the invention result from the disclosure and are explained in more detail in the following description with partial reference to the figures.

In contrast to solutions in which soft structures such as foams or comparable materials are used to reduce operating noise or sound, the invention is based on achieving a reduction in the emission of operating noise into the environment by configuring the routing of air volumes in solid structures.

The arrangement according to the invention for reducing noise can be arranged at a gas inlet of a blower ventilator. According to the invention, the arrangement has a connecting element (connection element) and at least one labyrinth element. The labyrinth element has a labyrinth structure which fills at least 40% of the volume of the labyrinth element. The labyrinth structure forms a plurality of parallel channels (ducts) with deflections in the at least one labyrinth element. The parallel channels are configured to guide air volumes and to influence sound propagation. The connecting element has a gas outlet which can be connected to a gas inlet of the blower ventilator. The connecting element has a gas inlet for the inflow of air volumes from an environment. The at least one labyrinth element has at least one inlet chamber for receiving inlet air volumes from the connecting element. The at least one labyrinth element has at least one outlet chamber for supplying inlet air quantities to the connecting element.

The connecting element is connected to the at least one labyrinth element in such a way that the air volumes from the environment flow into the labyrinth structure as inlet air volumes, flow through the plurality of channels of the at least one labyrinth element and flow into the gas inlet of the blower ventilator (forced-air breathing apparatus) as outlet air volumes via the connecting element and the gas outlet of the connecting element as breathing air volumes. The outlet chamber of the at least one labyrinth element is in fluid communication with the gas inlet of the blower ventilator, e.g., the gas inlet of the blower ventilator in the form of a flow channel extends through the gas outlet of the connecting element and seals the outlet chamber and/or the outlet of the labyrinth structure, so that the ambient air flowing through the gas inlet of the connecting element can only flow through the two inlet chambers, through the labyrinth structure, and through the outlet chamber. In an alternative embodiment, with two labyrinth elements on the connecting element, the connecting element includes internal structures to direct the flow accordingly. For example, the two labyrinth elements are flowed through serially in opposite directions, with the ambient air flowing through the outlet chamber, into the labyrinth structure, and then through the two inlet chambers of the first labyrinth element, subsequently through two flow channels within the connecting element to the two inlet chambers of the second labyrinth element, and then through the labyrinth structure and the outlet chamber of the second labyrinth element, and through the gas outlet of the connecting element. The internal structures of the connecting element may also direct the air flow through the two labyrinth elements in parallel. Many other embodiments may be provided as to directing air through the connecting element and the labyrinth element. The essential point is that the connecting element is configured in such a way that the ambient air flows through the labyrinth element with its labyrinth structure, as described above, whether in the case of one or multiple labyrinth elements connected to the connecting element. This achieves the noise reduction of the arrangement. The guidance of the flow within the connecting element to the labyrinth element or elements is particularly intended to accommodate different conditions such as connection possibilities or the size of the entire arrangement.

The sound propagation is influenced by the labyrinth structure in the labyrinth element in conjunction with the connecting element. The influence has the effect of reducing the radiation of operating noise to the environment (surrounding area), which is generated by the blower drive (fan drive) of the blower ventilation drive both at high and low air flow rates as a side effect of the air flow rate, by the structures with the channels with deflections.

The configuration of the labyrinth structure with the structures of channels with deflections in the at least one labyrinth element has the effect that the operating noises are directed, deflected and reflected in such a way that the sound level of these operating noises at the gas inlet of the connecting element is significantly reduced compared to the sound level at the blower. The dimensioning of the connecting element is selected such that a flow cross-section A_in at the gas inlet of the connecting element corresponds to a flow cross-section A_out at the at least one gas outlet of the connecting element. For the purposes of the invention, the flow cross-section A_in at the gas inlet and the flow cross-section A_out at the at least one gas outlet are of approximately (essentially) the same order of magnitude in terms of area. Furthermore, the shape of the flow cross-section A_in and the shape of the flow cross-section A_out are different, essentially the same or identical. The shape of the respective flow cross-section is, for example, round, rectangular, square, oval or elliptical.

The previously described dimensioning of the connecting element ensures that the connecting element and/or the at least one labyrinth element do not cause any significant pressure drop at the gas inlet of the blower ventilator for the inflow of air from the environment. Thus, the connecting element together with the at least one labyrinth element can be regarded as largely neutral with regard to an influence on the control and/or regulation of the blower in the blower ventilator in terms of pressure level and flow rates.

Preferred embodiments provide details of how the dimensions of the channels in the labyrinth element or inlet chamber and/or outlet chamber of the labyrinth element can be configured.

Thus, a sum of all flow cross-sections of the channels in the at least one labyrinth element can be selected such that it corresponds to the sum of the flow cross-sections of the at least one inlet chamber of the at least one labyrinth element.

Thus, a sum of all flow cross-sections of the channels in the at least one labyrinth element can be selected such that it corresponds to the sum of the flow cross-sections of the at least one outlet chamber of the at least one labyrinth element.

Thus, the sum of the flow cross-sections of the at least one inlet chamber of the at least one labyrinth element can be selected such that it corresponds to the sum of the flow cross-sections at the at least one gas inlet of the connecting element.

In practical implementation, these dimensions of the flow cross-sections have proven to be suitable orientation aids in order to achieve the best possible reduction of operating noise in adaptation to the frequency ranges of the noise or sound emissions generated by the blower drive through suitable configurations of the inlet chamber and/or outlet chamber of the labyrinth element.

Embodiments provide different configurations and numbers of labyrinth elements or closure elements arranged on the connecting element.

Thus, in a further preferred embodiment, it can be provided that at least two labyrinth elements or at least one closure element are arranged on the connecting element. A closure element corresponds to a labyrinth element in which no labyrinth structure is contained; the closure element is thus an empty spatial volume, as it were, which helps to reduce the operating noise by reflecting and deflecting the operating noise in the interaction between the connecting element and the labyrinth element.

Embodiments can be formed in which two labyrinth elements can be arranged opposite one another on the connecting element. Furthermore, embodiments can be formed in which a closure element and a labyrinth element can be arranged opposite one another on the connecting element.

Embodiments with different lengths L of the connecting element available for flow and different geometric shapes of the flow cross-section of the connecting element can be created.

Depending on the configuration and space situation at the gas inlet of the blower ventilator, the gas inlet and/or the gas outlet in the connecting element can be arranged relative to each other.

In a preferred embodiment, the arrangement of the connecting element and the two labyrinth elements, or the at least one labyrinth element and the closure element, is configured in such a way that the flow quantities flowing into the gas inlet of the connecting element from the environment and flowing out from the connecting element to the blower ventilator flow on two parallel axes.

The gas inlet and the gas outlet of the connecting element can be arranged on the same spatial axis.

The gas inlet and the gas outlet of the connecting element can be arranged with a horizontal offset to each other on two parallel spatial axes.

The gas inlet and the gas outlet of the connecting element can be arranged with a vertical offset to each other on two parallel spatial axes.

The gas inlet and the gas outlet of the connecting element can be arranged with a horizontal and vertical offset to each other on two parallel spatial axes.

Depending on the configuration and space situation at the gas inlet of the blower ventilator, the gas inlet and/or the gas outlet in the connecting element can be arranged in relation to or at an angle to the at least one labyrinth element or the two labyrinth elements arranged opposite each other.

In a preferred embodiment, the arrangement of the connecting element and the two labyrinth elements, or the at least one labyrinth element and the closure element, is such that the flow quantities which flow into the gas inlet of the connecting element from the environment and flow out from the connecting element to the blower ventilator form a 90° configuration in relation to the flow quantities which flow into and out of the labyrinth element or the closure element. This results in the inflowing flow quantities being deflected laterally by 90° directly after flowing in through the gas inlet and then flowing into the labyrinth element or the closure element and being deflected laterally by 90° when flowing out of the labyrinth element or the closure element and then flowing out through the gas outlet.

Depending on the configuration and space situation at the gas inlet of the forced ventilation device, round, rectangular, square, oval or elliptical cross-sections can form the basis for the configuration of the length L of the connecting element. In these embodiments, the dimensions between the length L of the connecting element and the cross-section of the connecting element can be easily implemented in practice.

In an advantageous way, the length L of a connecting element with a circular cross-section available for a flow through can be at least three times the internal diameter of the connecting element with a circular cross-section.

For example, the length of a connecting element with a square cross-section available for a flow can be at least three times a diagonal of the cross-section of the connecting element with a square cross-section.

For example, the length L of a connecting element with a rectangular cross-section available for flow can be at least three times a diagonal of the cross-section of the connecting element with a rectangular cross-section.

For example, the length L of a connecting element with an oval or elliptical cross-section available for a flow can be at least three times the larger half-axis of an ellipse or at least three times the diameter of an essentially round comparative geometry with an identical cross-section of the connecting element with an oval or elliptical cross-section.

The connecting element can be configured in such a way that a square of the length L of the connecting element available for flow is at least nine times the free flow cross-section within the connecting element.

For example, the connecting element can be configured as a channel with a largely symmetrical cross-section with a length-to-width ratio of approximately (essentially) 1:1.

The flow cross-sections of the at least one labyrinth element can have a round or square cross-section and the cross-section of the gas outlet can have a round or square cross-section.

The connecting element can also be configured as a channel with a largely asymmetrical cross-section with a length-to-width ratio of approximately (essentially) 2:1.

The flow cross-sections of the at least one labyrinth element can have a rectangular or oval cross-section and the cross-section of the gas outlet can have a rectangular or oval cross-section.

In preferred embodiments, the labyrinth structures can fill more than 50%, preferably more than 60%, of the volume of the labyrinth elements.

In further preferred embodiments, the channels can have a plurality of 90° deflections and/or a plurality of 180° deflections. These dimensions and configurations of the channels with the deflections and the filling of the space in the labyrinth elements have proven to be suitable in practical implementation in order to achieve the best possible reduction of operating noise in adaptation to the frequency ranges of the noise or sound emissions generated by the blower drive.

In preferred embodiments, the labyrinth structures can be configured and arranged in the at least one labyrinth element in such a way that in the transition from a flow cross-section at the inlet chamber to flow cross-sections of the plurality of parallel channels, there is a sudden or abrupt reduction of the flow cross-section at the respective duct of the plurality of parallel channels by a difference of at least a factor of 2. Abrupt or abrupt reductions in the flow cross-section of the channels cause effective reflections and multiple reflections for the noise when conducting noise and reduce the noise emissions that remain as a result.

In summary, the present invention makes it possible to effectively reduce the operating noise emitted by the blower ventilator to the environment.

In the following, exemplary embodiments of the invention are explained in more detail with reference to the figures, without limiting the generality of the inventive concept. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of an arrangement for reducing noise;

FIG. 2 is a schematic view showing a labyrinth element according to FIG. 1 in detail;

FIG. 3 is a schematic view showing a representation of connecting elements with two labyrinth elements.

### DESCRIPTION OF PREFERRED EMBODIMENTS

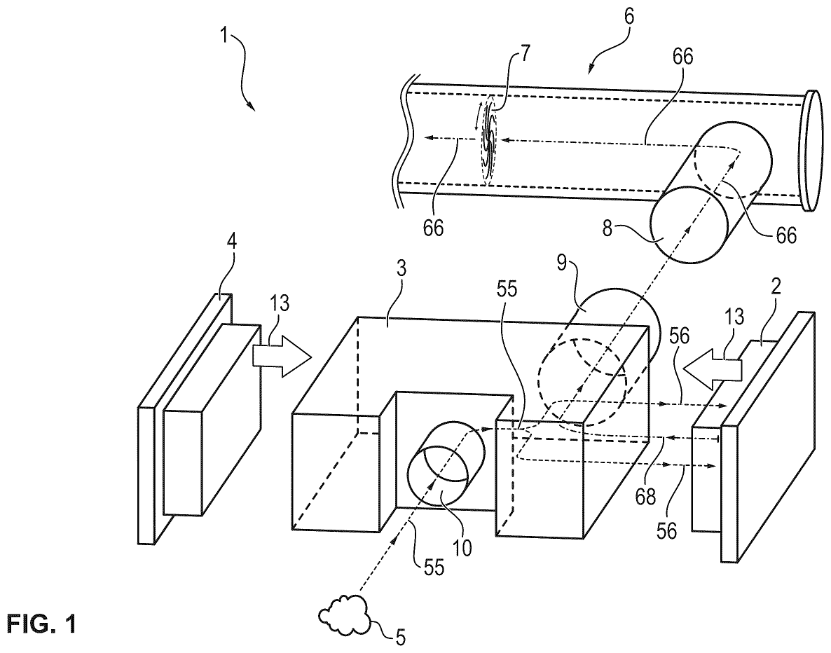

Referring to the drawings, FIG. 1 shows an arrangement 1 for reducing noise for a medical device for ventilating a living being with a blower ventilator 6 schematically indicated in the form of a blower drive unit. The blower ventilator 6 or the blower drive unit 6 has a radial blower 7 and a gas inlet 8. Other components of a blower ventilator 6 or ventilator, such as pneumatic connections, housing, valves, sensors, breathing system, hose lines, ventilation hoses, control unit or display and operating unit are not shown in FIG. 1, as they do not need to be explained in order to understand the sound reduction, but would impair the clarity of the drawing.

A connecting element 3 can be pneumatically coupled to the gas inlet 8 of the blower ventilator 6 by means of a gas outlet 9. Elements for sealing the coupling between gas inlet 8 and gas outlet 9 are present and dimensioned in the usual way, e.g. in the form of O-rings, but are not shown in order to maintain clarity of the drawing. The radial blower 7 conveys breathing air quantities 66 from the connecting element 3 via the gas outlet 9 and the gas inlet 8 into a breathing system of the blower ventilator 6. The blower ventilator then makes these breathing air quantities 66 available to a living being as part of controlled and/or supportive ventilation.

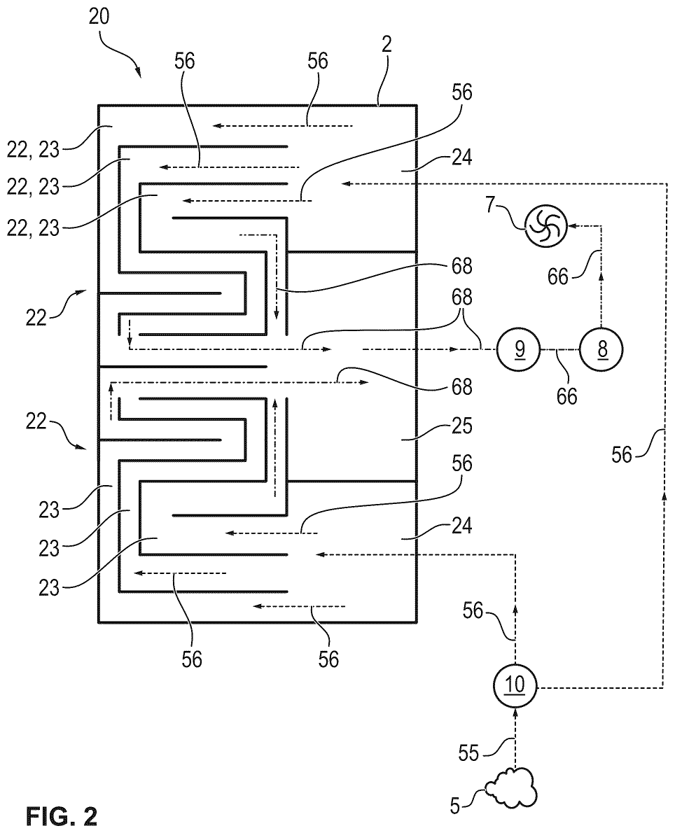

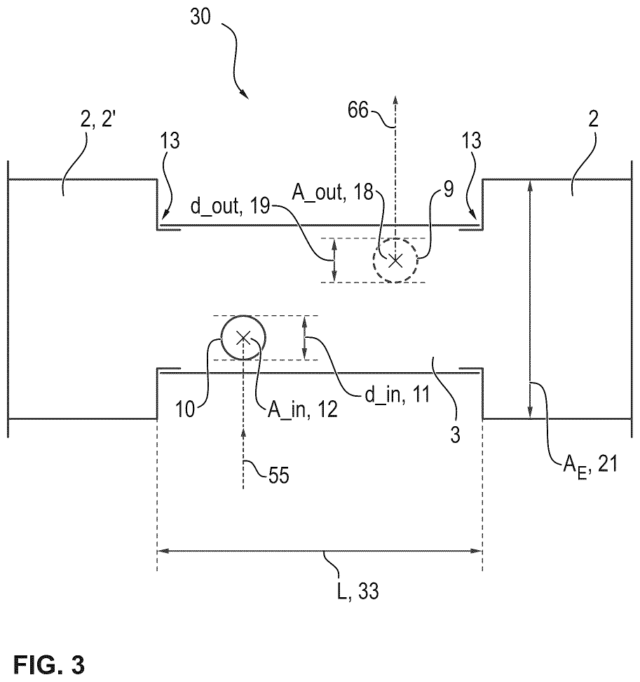

At least one labyrinth element 2 or 2′ (FIG. 3) with a labyrinth structure 22 (FIG. 2) forming channels 23 (FIG. 2), with at least one inlet chamber (24) and with at least one outlet chamber 25 (FIG. 2) can be coupled to the connecting element 3.

The arrows 13 are intended to illustrate the coupling of the labyrinth elements 2 or 2′ (FIG. 3) to the connecting element 3. For this, the connecting element 3 has connection seats at each side end allowing connection and disconnection of a labyrinth element (2 or 2′). The respective connection seat receives the respective labyrinth element 2 or 2′.

In this FIG. 1, a closure element 4 is shown on the connecting element 3 (at the connection seat at the left side in FIG. 1) opposite the labyrinth element 2. The closure element 4 does not have a labyrinth structure and thus only provides a function of a sealing lateral closure of the connecting element 3. Sealing elements between connecting element 3, labyrinth element 2 and closure element 4 are provided in the usual manner, but are not shown in this FIG. 1 for the sake of clarity.

However, a wide variety of other variants of the arrangement 1 can also be configured to reduce noise for a blower ventilator 6, which have at least two labyrinth elements 2, 2′ (FIG. 3), for example in an opposing arrangement with a centrally arranged connecting element 3.

Ambient air quantities 55 enter the connecting element 3 from an environment 5 via a gas inlet 10 and are provided as inlet air quantities 56 of at least one inlet chamber 24 (FIG. 2) arranged in the labyrinth element 2. In the labyrinth element 2, the ambient air quantities 55 are guided through the channels 23 (FIG. 2) of the labyrinth structure 22 (FIG. 2) and via at least one outlet chamber 25 (FIG. 2) as outlet air quantities 68 back into the connecting element 3 and from there via the gas outlet 9 of the connecting element 3 then as breathing air quantities 66 to the gas inlet 8 of the blower ventilator (forced-air breathing apparatus) 6. FIG. 1 shows that the flow quantities 55 after the gas inlet 10 in the connecting element 3 flow at right angles from the environment 5 as flow quantities 56 into the labyrinth element 2. FIG. 1 shows that the flow quantities 68 flow from the labyrinth element 2 in the connecting element 3 at right angles as flow quantities 68 into the gas outlet 9.

In FIG. 1, the gas inlet 10 and gas outlet 9 of the connecting element 3 are arranged in an exemplary configuration situation with a horizontal and vertical offset to each other on two parallel spatial axes. Alternative constructive situations in which only a horizontal offset or only a vertical offset are configured are, however, included in the core idea of the configuration of the connecting element 3, but are not shown in FIG. 1 in order to maintain a simplified schematic and clear representation. Suitable positioning of the connecting element 3 at the gas inlet 8 of the blower ventilator 6 results from the shape and dimensions of the blower ventilator 6 with the aim of achieving the most space-saving and flow-optimized arrangement possible.

Details of the components and functionality of the labyrinth element 2 are explained in more detail below with reference to FIGS. 2 and 3. Identical elements in FIGS. 1, 2, 3 are designated in FIGS. 1, 2, 3 with identical reference numbers.

FIG. 2 shows a schematic representation 20 of the labyrinth element 2 with labyrinth structure 22 and channels 23 extending through the labyrinth element 2, which guide the inflow 56, through-flow and outflow 68 of air quantities through the labyrinth element 2. The inflow 56 of ambient air quantities 55 from the environment 5 takes place in this configuration example according to FIG. 2 from the connecting element 3 (FIG. 1) via the gas inlet 10 through two inlet chambers 24. The outflow 68 of outlet air quantities back to the connecting element 3 (FIG. 1) and then via the gas outlet 9 of the connecting element 3 (FIG. 1) to the gas inlet 8 of a blower ventilation device 6 up to the radial blower 7 takes place in this configuration example according to FIG. 2 by means of an outlet chamber 25.

However, embodiments with several inlet chambers and/or several outlet chambers are also possible; the variant shown in this FIG. 2 with two inlet chambers 24 and one outlet chamber 25 is adapted to the rectangular outer contours of the labyrinth element 2 and the space-saving configuration of the assembly of labyrinth elements 2, 2′ (FIG. 1, FIG. 3), the connecting element 3 (FIG. 1) with the gas inlet 8 of the blower ventilator 6 (FIG. 1) to form an arrangement 1 (FIG. 1) for reducing noise at the gas inlet 8 of a blower ventilator 6 (FIG. 1).

FIG. 3 shows a representation 30 of connecting element 3 and two labyrinth elements 2, 2′. The gas inlet 10 is shown schematically with an inlet diameter d_in 11 and an inlet cross-section A_in 12 on the connecting element 3. The gas outlet 9 is shown schematically with an outlet diameter d_out 19 and an outlet cross-section A_out 18 on the connecting element 3. The distance between the two labyrinth elements 2, 2′ is shown schematically as length L 33. A sum cross-section A_E 21—sum of the cross sections of the plurality of channels 23 (FIG. 2) of the labyrinth elements (2, 2′)—is also indicated. In addition, the ambient air volume 55 at the gas inlet 10 and the breathing air volume 66 at the gas outlet 9 of the connecting element 3 are schematically indicated.

Based on FIG. 3 with references to FIGS. 1 and 2, some exemplary advantageous dimensional relationships between flow cross-sections A 12, 18, diameters d 11, 19 and lengths L 33 will now be illustrated in more detail.

It is essential, for example, that the inlet cross-section A_in 12, outlet cross-section A_out 18 on the connecting element 3 and the sum cross-section A_E 21 of all channels 23 (FIG. 2) of the labyrinth elements (2, 2′) are of approximately the same order of magnitude.

Furthermore, a configuration is preferred in such that a square of the length L 33 of the connecting element 3 available for a throughflow has at least nine times the free flow cross-section given within the connecting element 3.

Furthermore, in the case of a connecting element with a circular diameter, it is advantageous if the ratio of the length L 33 is at least three times the internal diameter of the connecting element 3 associated with the length L 33.

Furthermore, it is advantageous if the labyrinth structures 22 (FIG. 2) fill more than 40%, preferably more than 50%, more preferably more than 60% of the volume of the labyrinth elements 2, 2′.

Furthermore, it is advantageous if the channels 23 have a plurality of 90° deflections and/or a plurality of 180° deflections.

These dimensioning aids can also be transferred to other geometries and cross-sectional shapes of the connecting element 3 by means of conventional area calculations and conversions, for example to oval, elliptical, rectangular or square cross-sectional shapes of the connecting element and geometries of the gas inlet 10 or the gas outlet 9 of the connecting element 3 that deviate from the round shape, as well as to configurations with the square/rectangular shape of the labyrinth elements 2, 2′.

While specific embodiments of the invention have been shown and described in detail to illustrate the application of the principles of the invention, it will be understood that the invention may be embodied otherwise without departing from such principles.

### REFERENCE NUMBER LIST

1 Arrangement for noise reduction
2, 2′ Labyrinth element with labyrinth structure
3 Connecting element
4 Closing element, cover
5 Surroundings
6 Blower ventilator, blower drive unit
7 Centrifugal blower
8 Gas inlet of a blower ventilator
9 Gas outlet of the connecting element
10 Gas inlet of the connecting element
11 Diameter d_in of the gas inlet of the connecting element
12 Cross-section A_in of the gas inlet of the connecting element
13 Assembly,
18 Cross-section A_out of the gas outlet of the connecting element
19 Diameter d_out of the gas outlet of the connecting element
20 Representation of the labyrinth arrangement in the labyrinth element
21 Summary cross-section A_E of the channels of the labyrinth elements
22 Labyrinth structure
23 Channels
24 Inlet chamber on the labyrinth element
25 Outlet chamber on the labyrinth element
30 Representation of connecting element and labyrinth element
33 Length L of the connecting element
55 Ambient air volumes from the surroundings
56 Inflow into the inlet chamber on the labyrinth element
66 Breathing air volumes from outlet chamber, outflow
68 Outlet air volume from outlet chamber

What is claimed is:

1. An arrangement for reducing noise at a gas inlet of a blower ventilator, the arrangement comprising:
a connecting element, the connecting element comprising:
a gas outlet which is configured to be connected to a gas inlet of the blower ventilator; and
a gas inlet configured for an inflow of air quantities from an environment;
a labyrinth element, the labyrinth element comprising:
an inlet chamber configuration comprising an inlet chamber or a plurality of inlet chambers, the inlet chamber configuration being configured to receive inlet air quantities from the connecting element;
an outlet chamber configuration comprising an outlet chamber or a plurality of outlet chambers, the outlet chamber configuration being configured to provide outlet air quantities to the connecting element;
a labyrinth structure that fills more than 40% of a volume of the labyrinth element, wherein the labyrinth structure forms a plurality of parallel channels with deflections in the labyrinth element, wherein the parallel channels are configured to guide air volumes and to influence sound propagation, wherein the connecting element is connected to the labyrinth element such that the air quantities from the environment flow into the inlet chamber configuration of the labyrinth element as inlet air quantities, flow through the plurality of channels of the labyrinth element and through the outlet chamber configuration of the labyrinth element and flow as outlet air quantities via the connecting element and the gas outlet of the connecting element as breathing air quantities into the gas inlet of the blower ventilator, wherein a flow cross-sectional area at the gas inlet of the connecting element corresponds to a flow cross-sectional area of the gas outlet of the connecting element.

2. An arrangement according to claim 1,
wherein a sum of all flow cross-sections of the channels in the labyrinth element corresponds to a sum of flow cross-sections of the inlet chamber configuration of the labyrinth element,
wherein the sum of all flow cross-sections of the channels in the labyrinth element corresponds to a sum of flow cross-sections of the outlet chamber configuration of the labyrinth element,
wherein the sum of the flow cross-sections of the inlet chamber configuration of the labyrinth element corresponds to the sum of the flow cross-sections at the gas inlet of the connecting element.

3. An arrangement according to claim 1, further comprising another labyrinth element or a closure element, wherein the labyrinth element and the other labyrinth element are arranged on the connecting element or the labyrinth element and the closure element are arranged on the connecting element.

4. An arrangement according to claim 1, further comprising another labyrinth element, wherein the arrangement comprises two labyrinth elements, each with the labyrinth structure, and each labyrinth element is arranged opposite to the other labyrinth element on the connecting element.

5. An arrangement according to claim 3, wherein the labyrinth element with labyrinth structure and the closure element without labyrinth structure are arranged opposite to one another on the connecting element.

6. Arrangement according to claim 1,
wherein the connecting element is configured with a circular cross-section available for a through-flow and a length of the circular cross-section available for the through-flow is at least three times an internal diameter of the circular cross-section available for the through-flow, or
wherein the connecting element is configured with a square cross-section available for a through-flow and a length of the square cross-section available for the through-flow is at least three times a diagonal of the square cross-section available for the through-flow, or
wherein the connecting element is configured with a rectangular cross-section available for a through-flow and a length of the rectangular cross-section available for the through-flow is at least three times a diagonal of the rectangular cross-section available for the through-flow, or
wherein the connecting element is configured with an oval or elliptical cross-section available for a through-flow and a length of the oval or elliptical cross-section available for a through-flow is at least three times a larger half-axis of an ellipse of the elliptical cross-section available for the through-flow or at least three times a diameter of a substantially round comparative geometry with an identical cross-section of the oval or elliptical cross-section available for the through-flow.

7. An arrangement according to claim 1, wherein the connecting element is configured such that a square of a length of the connecting element available for a through flow is at least nine times a free flow cross-section provided by the connecting element.

8. An arrangement according to claim 1,
wherein the connecting element is configured as a channel with an essentially symmetrical cross-section with a length-to-width ratio of essentially 1:1 and wherein the flow cross-sections of the labyrinth element have a round or square cross-section and wherein the cross-section of the gas outlet has a round or square cross-section, or
wherein the connecting element is configured as a flat surface channel with an asymmetrical cross-section with a length-to-width ratio of more than 2:1 and wherein the flow cross-sections of the labyrinth element have a rectangular or oval cross-section and wherein the cross-section of the gas outlet has a rectangular or oval cross-section.

9. An arrangement according to claim 1, wherein the labyrinth structure fills more than 50% of the volume of the labyrinth elements and wherein the channels have a plurality of 90° deflections and/or a plurality of 180° deflections.

10. An arrangement according to claim 1, wherein the labyrinth structure is formed and arranged in the labyrinth element such that in a transition, from a flow cross-section at the inlet chamber to flow cross-sections of the plurality of parallel channels, there is a sudden or abrupt reduction of the flow cross-section at the respective channel of the plurality of parallel channels by a difference of at least a factor of 2.
